(19) United States
(12) Patent Application Publication
(10) Pub. No.: US 2005/0096683 A1
(43) Pub. Date: May 5, 2005

(54) Using thinner laminations to reduce operating temperature in a high speed hand-held surgical power tool

(75) Inventors: Rob Ellins, Euless, TX (US); Christian Fleury, (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/698,114

(22) Filed: Nov. 1, 2003

(51) Int. Cl. A61B 17/32

(52) U.S. Cl. 606/170

(57) ABSTRACT

A surgical instrument having an electric motor is discussed. The motor includes a motor output member, a driven member and a driving member. The driven member is coupled to the motor output member. The driving member includes a winding and a magnetically conductive portion comprising a plurality of laminations having thicknesses of less than 0.25 mm. The driving member, or at least the magnetically conductive portion thereof, is disposed proximate the driven member such that energizing the driving member imparts motion to the driven member.

### RELATED APPLICATIONS

This application is related to the commonly-assigned and concurrently filed U.S. Patent Application entitled “ELECTRIC MOTOR HAVING NANOCRYSTALLINE ALLOY COMPONENT FOR USE IN SURGICAL PROCEDURE”, Attorney Docket No. 31849.41, having Thierry Bieler, Christian Koechli, Laurent Cardoletti, and Christian Fleury named as inventors, which concurrently filed application is incorporated herein by reference in its entirety.

This application is related to the commonly-assigned and concurrently filed U.S. Patent Application entitled “SMALL HAND-HELD MEDICAL DRILL,” Attorney Docket No. P-11714.00US, having Christian Fleury, Rob Ellins, Manfred Lutdi, and Thierry Bieler named as inventors, which concurrently filed application is incorporated herein by reference in its entirety.

### BACKGROUND

This application relates to hand-held surgical tool systems powered by electrical motors.

An ideal hand-held surgical power tool system would be lightweight and would generate sufficient power and be sufficiently small for the task at hand. However, producing a power tool system with such features can be difficult. In part, this is due to the fact that electrical motors produce heat. As the power of a motor increases, the heat generated by the motor generally increases.

Motor design and configuration begin to address this problem. For example, direct current (dc) motors are capable of operating at high efficiencies at extremely high speeds, yet heat generation remains a problem. One way to reduce heat generation in a motor is to adopt a brushless configuration, such as in a brushless dc motor, in which no electrical or mechanical contact is required between the source of electrical power and the rotating component of the motor. A brushless dc motor typically includes an external, slotted or non-slotted stator structure having windings therein. The motor also includes a rotor having a shaft and a hub assembly comprising a magnetic structure, at least in part. In general, the rotor rotates within an inner cavity of the stator, although in some applications the rotor may be disposed outside of the stator. In both scenarios, brushless dc motors produce output torque via interaction between the stator and the rotor due to a magnetic field produced by the permanent magnet of the rotor and/or a magnetic field due to an electrical current in the stator (windings).

Brushless dc motors and other conventional dc motors are employed to produce mechanical power or torque from electric power. However, conventional dc motors do not perform this conversion efficiently. Losses arising as a motor produces mechanical power in response to electric power result in limitations in power, torque and speed. These losses can generally be classified into three categories: (1) load sensitive losses dependent on generated torque; (2) speed sensitive losses dependent on motor speed; and (3) pulse-width modulation (PWM) losses dependent on the quality of the current supply employed to drive the motor.

The load or torque sensitive losses are generally limited to windings losses which are proportional to the product of the square of the current through the windings and the resistance of windings. Speed sensitive losses (e.g., core or iron losses due to Eddy currents and hysteresis, windage and friction) act as a velocity dependent torque opposite the output torque of the motor. PWM losses are attributable to Eddy currents in the magnetic structure caused by the power supply. Such Eddy currents can deleteriously result in a high frequency current oscillation in the windings.

Eddy currents are phenomena caused by a variation of magnetic field through an electrically conductive medium. In the case of brushless dc motors, the medium that experiences the change of magnetic field in which a voltage potential is induced is the magnetically conductive part of the stator. The rotation of the rotor or the current variation in the windings induce a voltage in the magnetically conductive part of the stator, which results in the creation of Eddy currents. These currents can have a significant heating effect on the motor, particularly when operating at high speeds or with high a current ripple in the windings. Thus, higher speeds generally create more heat. During use, hand-held surgical tool systems may become too hot for a surgeon to continue operation of the power tool.

Decreasing the power or speed of the motor is one way to reduce heat, but this is often not an acceptable option. Another way to reduce the temperature of a hand-held surgical tool system during use is to incorporate an active cooling system in the tool. Such tool systems may include an air or liquid cooling system. However, the introduction of an active cooling system into a hand-held tool tends to increase overall size and weight of the system.

A possibility, which has not generally been adapted for use in surgical power tool systems, is to reduce the thickness of laminations of the magnetic material forming the stator. The magnetically conductive portion of the stator is not generally formed of a solid block, but rather is typically formed of a thin stack of sheets called laminations. Typical lamination thickness in surgical power tool systems is between about 0.25 mm and 1 mm, depending on the intended use. Reduction in lamination thickness has been known to result in decreased losses thought to be due to Eddy currents. Such reduced losses, which are believed to be due to one aspect of loss, i.e. Eddy currents, correlate to decreased heat. However, it is uncertain to what extent further reduction in lamination thickness will affect overall heat generation in a surgical power tool system.

Because of the relationship between size, power and heat generation, it is difficult as a practical matter to produce a hand-held surgical tool system with a powerful motor in a size small enough to be useful to a surgeon over periods of extended use. For example, if size were not a concern one could readily increase power of a motor by increasing size of the motor, and active cooling systems could readily be adapted. However, size is a practical consideration. As such, production of a high-speed hand-held surgical device of sufficiently small size that can be operated over periods of time without excessive heat generation has continued to be a challenge.

### SUMMARY

The present disclosure provides a description of a hand-held device and associated motor having desirable size, power or speed, and heat characteristics for use in surgical applications over extended periods of time. In one aspect, the device is of a similar size and power as currently available devices but may be used for longer periods of time without excessive heating.

In an embodiment, the invention provides a surgical instrument. The surgical instrument includes a housing, an electrical power source and an output shaft extending from the housing. The instrument also includes a rotor coupled to the output shaft. A stator having a winding selectively connectable to the electrical power source and a magnetically conductive portion is disposed about the rotor. At least a portion of the stator comprises a plurality of laminations. One or more or each of the laminations has a thickness of less than about 0.25 mm. In an embodiment, one or more or each of the laminations has a thickness less than or equal to about 0.2 mm. In an embodiment, one or more or each of the laminations has a thickness less than or equal to about 0.15 mm. In an embodiment, one or more or each of the laminations has a thickness less than or equal to about 0.1 mm. Selectively connecting the electrical power source and the stator winding(s) imparts rotary motion to the output shaft via the rotor.

An embodiment of the invention provides an electrical motor including a motor output member, a driven member and a driving member. The driven member is coupled to the motor output member. The driving member includes a winding and a magnetically conductive portion disposed proximate the driven member such that energizing the driving member imparts motion to the driven member. The magnetically conductive portion comprises a plurality of laminations. One or more or each of the laminations has a thickness of less than about 0.25 mm. In an embodiment, one or more or each of the laminations has a thickness less than or equal to about 0.2 mm. In an embodiment, one or more or each of the laminations has a thickness less than or equal to about 0.15 mm. In an embodiment, one or more or each of the laminations has a thickness less than or equal to about 0.1 mm.

Motors and instruments as described herein may provide several advantages. For example, when used in surgical applications, the instruments described herein can reduce surgery time and increase ease of surgery. Because motors and instruments including the motors as described herein produce less heat without sacrificing power, a surgeon will require less breaks during surgery to allow the instrument to cool down. The surgeon may require no breaks at all during surgery. Because motors and instruments including the motors as described herein do not result in increased size, a surgeon will experience less hand fatigue. These and other advantages will be evident to those skilled in the art based on the description herein.

The foregoing has outlined preferred and alternative features of several embodiments so that those skilled in the art may better understand the detailed description that follows. Additional features will be described below that further form the subject of the claims herein. Those skilled in the art should appreciate that they can readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 illustrates a perspective environmental view of a surgical instrument for the dissection of bone and other tissue according to aspects of the present invention.

FIG. 2 illustrates a perspective view of one embodiment of the surgical instrument shown in FIG. 1.

FIG. 3 illustrates a perspective view of one embodiment of an electric motor constructed according to aspects of the present invention.

FIG. 4 illustrates a perspective view of another embodiment of the electric motor shown in FIG. 3.

FIG. 5 illustrates a perspective view of another embodiment of an electric motor constructed according to aspects of the present invention.

FIG. 6 illustrates an exploded perspective view of one embodiment of an electric disc motor constructed according to aspects of the present invention.

FIG. 7 illustrates an elevation view of one embodiment of an electric linear motor constructed according to aspects of the present invention.

FIG. 8 is a side view of a section of portion of a surgical instrument according to aspects of the present invention.

FIG. 9 is a graph of thermal cross comparison data of surgical instruments having motors with stator laminations of differing thicknesses.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over, on or coupled to a second feature in the description that follows may include embodiments in which the first and second features are formed in direct contact, and may also include embodiments in which additional features may be formed interposing the first and second features, such that the first and second features may not be in direct contact.

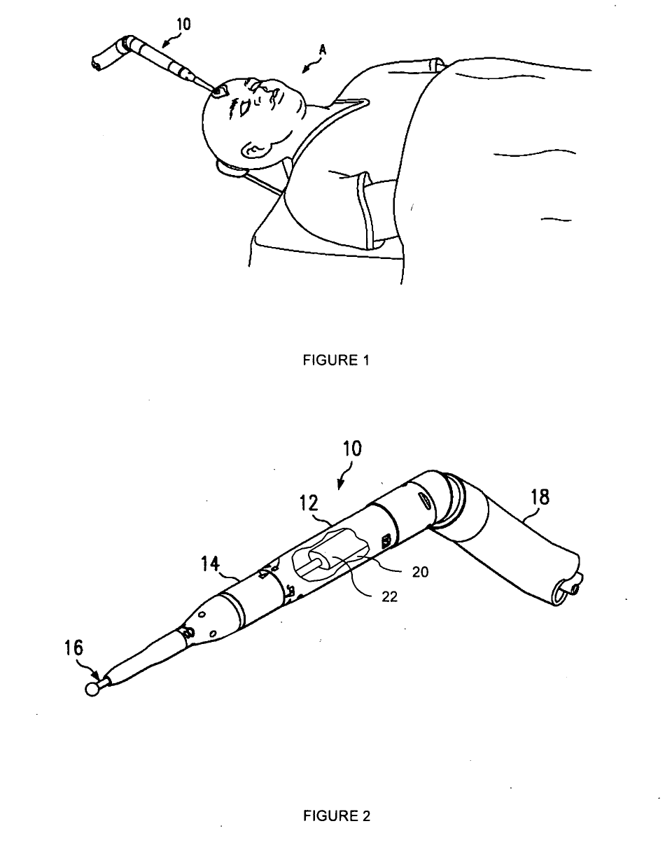

Referring to FIG. 1, illustrated is a perspective environmental view of one embodiment of a surgical instrument 10 for the dissection of bone and other tissue according to aspects of the present disclosure. The surgical instrument 10 is shown operatively associated with a patient A for performing a craniotomy. It will become apparent to those skilled in the art that the described instrument is not limited to any particular surgical application but has utility for various applications in which it is desired to dissect bone or other tissue. Additional applications include:
1. Arthroscopy—Orthopaedic
2. Endoscopic—Gastroenterology, Urology, Soft Tissue
3. Neurosurgery—Cranial, Spine, and Otology
4. Small Bone—Orthopaedic, Oral-Maxiofacial, Ortho-Spine, and Otology
5. Cardio Thoracic—Small Bone Sub-Segment
6. Large Bone—Total Joint and Trauma
7. Dental.

Referring to FIG. 2, illustrated is a perspective view of one embodiment of the surgical instrument 10 shown in FIG. 1. The surgical instrument 10 is illustrated to generally include a motor assembly 12, an attachment housing 14 and a surgical tool 16. The attachment housing 14 may provide a gripping surface for use by a surgeon and may also shield underlying portions of the instrument 10 during a surgical procedure. In a preferred embodiment, the surgical tool 16 is a cutting tool or dissection tool, although the type of tool is not essential to implementing the present disclosure.

The surgical instrument 10 is shown connected to a power cord assembly 18 for providing a source of electrical power to the motor assembly 12. It is further understood, however, that embodiments of the surgical instrument 10 according to aspects of the present disclosure will have equal application for a battery powered surgical instrument, such that the surgical instrument 10 may alternatively or additionally include disposable and/or rechargeable batteries. In such embodiments, the batteries may be housed within the motor assembly 12, or may be a separate, discrete component or subassembly. For example, the power cord assembly 18 shown in FIG. 2 may alternatively be a battery module containing one or more batteries.

The attachment housing 14 is adapted and configured to engage the motor assembly 12. The surgical tool 16 may be inserted into attachment housing 14 for engaging with the motor assembly 12. The motor assembly 12 includes an internal cavity 20 adapted and configured to contain a motor 22. Embodiments of the motor 22 are described in further detail below. In general, the motor 22 is coupled to the surgical tool 16 such that rotary or linear motion of the motor 22 may be imparted to the surgical tool 16.

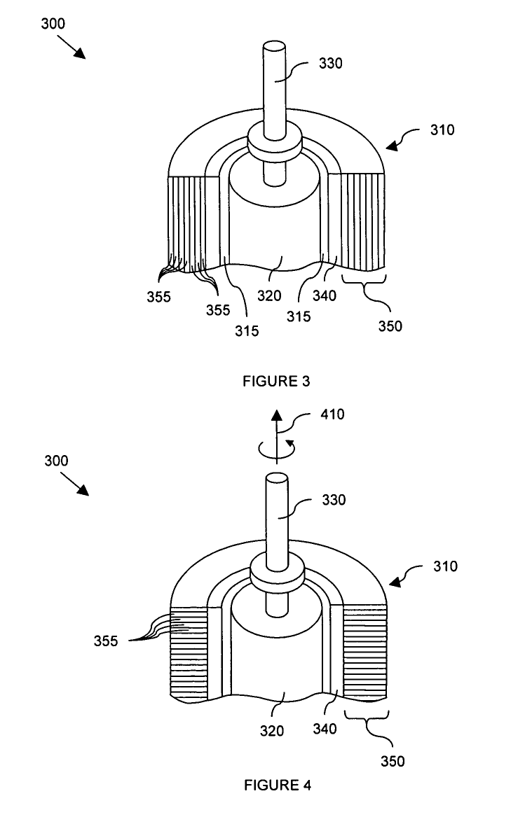

Referring to FIG. 3, illustrated is a perspective view of one embodiment of an electric motor 300 constructed according to aspects of the present disclosure. The electric motor 300 may be implemented for surgical environments, including those represented by FIGS. 1 and 2 and the corresponding description above. The electric motor 300 includes a stator 310, a rotor 320 and an output shaft 330 coupled to the rotor 320. In general, the rotor 320 is disposed within the cavity formed by the stator 310, such that the rotor 320 may rotate within the stator 310 in response to electric and/or magnetic fields generated by the stator 310 and/or the rotor 320.

The rotor 320 comprises a magnet of a magnetic component and may be formed by machining, casting, molding and/or other processes. Any magnet material may be used. For example, neodymium iron boron or samarium cobalt may be used as magnetic material. In one embodiment, the output shaft 330 and the rotor 320 are integrally formed. As discussed above, the output shaft 330 may also be configured to engage a surgical tool. For example, the output shaft 330 may include half of a pin/socket coupling or other means for rigidly but detachably securing a surgical tool. However, any conventional or future-developed output shaft 330, surgical tool and means for coupling thereof may be employed within the scope of the present disclosure.

The stator 310 includes at least one winding 340 coupled to a magnetically conductive portion 350. The winding(s) 340 may be of conventional composition and manufacture, such as a plurality of electrically conductive coils. However, the scope of the present disclosure does not limit the particular nature of the winding(s) 340, such that any conventional or future-developed windings may be employed according to aspects of the present disclosure. The winding(s) 340 are electrically insulated from the magnetically conductive portion 350. The winding(s) 340 may be selectively connectable to an electrical power source, such as the power cord/battery assembly 18 shown in FIG. 1, such as by an electrical switch.

The magnetically conductive portion 350 may comprise any suitable magnetically conductive material. In an embodiment, the magnetically conductive portion 350 comprises an alloy, such as an iron-based alloy. Iron-based alloys include iron-nickel alloys, iron-cobalt alloys, iron-cobalt-vanadium alloys, iron-nickel cobalt alloys, cobalt-iron alloys, and the like. The ratio of iron in an iron alloy may be changed to affect the properties of the alloy. Thus, a particular alloy most suitable for the intended use may be selected. In an embodiment, the alloy is an iron-nickel alloy. The iron-nickel alloy may contain any suitable percentage of iron and nickel. In an embodiment, the iron-nickel alloy comprises between about 45% and about 55% iron and between about 45% and about 55% nickel. The alloy may be a nanocrystalline alloy.

As shown in FIG. 3, the magnetically conductive portion 350 may comprise a plurality of laminations 355 each concentric to the winding 340 (and, thus, also concentric to the rotor 320, in the illustrated embodiment). The laminations 355 may each have a thickness less than about 0.25 mm. Employing a lamination 355 having a thickness of less than about 0.25 mm as at least a portion of the magnetically conductive portion 350 may reduce the generation of Eddy currents within the stator. Accordingly, losses conventionally deleterious to the efficiency and other performance characteristics of electric motors may be substantially reduced or eliminated by forming at least a portion of the stator 310 from a lamination having a thickness of less than about 0.25 mm. Thus, the electric motor 300 of the present disclosure may experience reduced losses at higher speeds compared to conventional motors. In one embodiment, the electric motor 300 operates at speeds ranging between about 100 rpm and about 100,000 rpm. In an embodiment, one or more of the laminations 355 have a thickness of less than or equal to about 0.2 mm. In an embodiment, one or more of the laminations 355 have a thickness of less than or equal to about 0.15 mm. In an embodiment, one or more of the laminations 355 have a thickness of less than or equal to about 0.1 mm. In an embodiment, the thickness of the laminations 355 ranges from between about 100 nm and about 100 μm. Of course, any individual or aggregate thickness of the layers 355 is within the scope of the present disclosure, provided that an individual animation 355 has a thickness of less than about 0.25 mm. In an embodiment, each lamination 355 has substantially the same thickness.

The laminations 355 may be formed by any suitable process, which are well-known in the art. For example, laminations may be formed from ribbon-shaped alloy material, such as that available from Imphy Ugine Precision, headquartered in La Defense, France, and Vacuumschmelze GmbH & Co. KG of Hanau, Germany. The ribbon-shaped alloy material may be punched into lamination sheets of a size and design suitable for the desired motor. The lamination sheets may then be annealed to optimize magnetically conductive characteristics for the intended use of the motor. Annealing typically consists of heating the lamination sheets to an elevated temperature. Conditions such as time, temperature, dew point, and atmosphere conditions may be varied to achieve desired magnetic characteristics. A surface oxide layer is preferably developed on the laminations 355. The surface oxide layer acts as an insulator and will provide resistance to Eddy current flow between the laminations. The annealed lamination sheets may be stacked to the desired height (core length) and held together by bolting, welding, or other means of interlocking to form at least a portion of the magnetic portion 350 of the stator 310. When preparing laminations 355 less than about 0.25 mm thick, care should be taken to not to deform the laminations, particularly after annealing.

Referring to FIG. 4, illustrated is a perspective view of another embodiment of the electric motor 300 shown in FIG. 3. In general, the embodiments shown in FIGS. 3 and 4 may be substantially similar. However, in contrast to the concentric nature of the laminations 355 of the magnetically conductive portion 350 shown in FIG. 3, the laminations 355 of the embodiment shown in FIG. 4 are substantially orthogonal to the axis of rotation 410 of the rotor 320. In other words, the laminations 355 may be radially stacked, as shown in FIG. 3, or axially stacked, as shown in FIG. 4. Of course, any other variation of orientation of the laminations 355 relative to the axis of rotation 410 of the rotor 320 may be employed in a motor, and the orientation of the laminations 355 may vary within a magnetically conductive portion 350 of a stator 310.

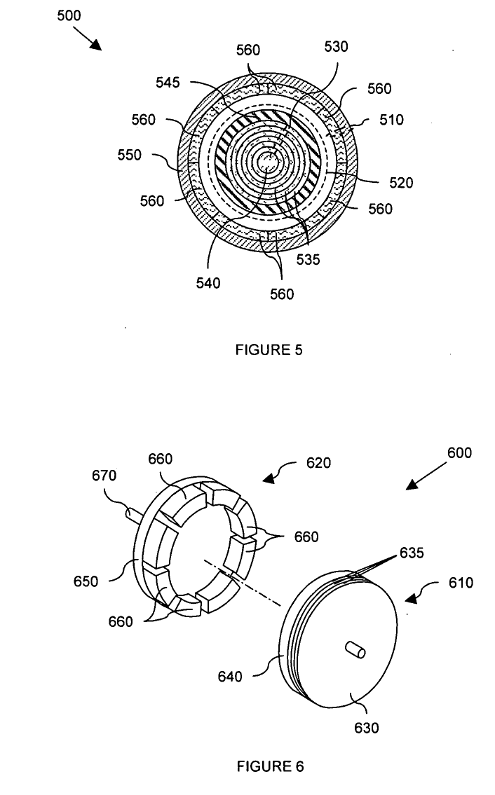

Referring to FIG. 5, illustrated is a plan view of another embodiment of an electric motor 500 constructed according to aspects of the present disclosure. In general, the electric motor 500 shown in FIG. 5 may be substantially similar to the electric motor 300 shown in FIG. 3. However, in contrast to the internal nature of the rotor 320 shown in FIG. 3, the electric motor 500 includes an external rotor 510. That is, the rotor 510 is disposed and configured to rotate about an internal stator 520. The stator 520 may be substantially similar in composition and manufacture to the stator 310 shown in FIG. 3. For example, the stator 520 includes a magnetically conductive portion 530 comprising a plurality of laminations 535. The laminations 535 may be formed around a core 540, which may be also be employed for connecting the electric motor 500 to surrounding structure (e.g., interior structure of the motor assembly 12 shown in FIG. 1). Moreover, as with the embodiments discussed above with reference to FIGS. 3 and 4, although FIG. 5 illustrates the laminations 535 as being radially stacked, the laminations 535 may also be axially stacked, stacked in an orientation between axial and radial, combinations thereof, etc. The stator 520 also includes at least one winding 545 disposed around the magnetically conductive portion 530.

The external rotor 510 may include a structural member 550 and one or magnets or magnetic components 560 (hereafter collectively referred to as the magnetic components 560) formed on or otherwise coupled to an interior surface of the structural member 550. The inner diameter of the external rotor 510 is configured such that the orientation of the magnetic components 560 relative to the internal stator 520 provides the desired interaction between the electric and/or magnetic field generated by the magnetic components 560 and/or the stator 520. In response to this interaction, the external rotor 510 will rotate around the internal stator 520, possibly at speeds up to about 1,000,000 rpm.

Referring to FIG. 6, illustrated is an exploded perspective view of another embodiment of an electric motor 600 constructed according to aspects of the present disclosure. The electric motor 600 includes a substantially disc-shaped stator 610 and a substantially disc-shaped rotor 620. The stator 610 includes a magnetically conductive portion 630 comprising a plurality of laminations 635, as in the embodiments described above. The stator 610 also includes at least one conventional or future-developed winding 640 located around the circumference of the magnetically conductive portion 630. The winding(s) 640 may also or alternatively be located on or recessed within a surface of the magnetically conductive portion 630 facing the rotor 620.

The rotor 620 includes a structural portion 650 having one or more magnets or magnetic components 660 (hereafter collectively referred to as the magnetic components 660) adhered or otherwise coupled to a surface of the structural portion 650 facing the stator 610. As shown in FIG. 6, the magnetic components 660 may collectively form a substantially disc-shaped annulus. The rotor 620 may also include an output shaft 670 coupled to or formed integrally with the structural portion 650, wherein the output shaft 670 may be substantially similar to the shaft 330 described above with reference to FIG. 3.

The embodiment shown in FIG. 6 may be particularly advantageous in applications in which higher torque and lower speeds are desired.

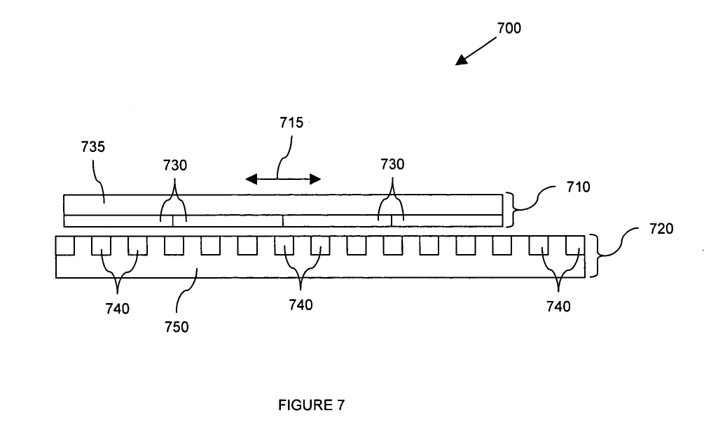

Referring to FIG. 7, illustrated is an elevation view of another embodiment of an electric motor 700 constructed according to aspects of the present disclosure. However, whereas the embodiments of the electric motors discussed above generally contemplate rotary motors, the electric motor 700 shown in FIG. 7 contemplates a linear motor. Apart from this distinction, the electric motor 700 may be substantially similar to the electric motor 300 shown in FIG. 3.

For example, the electric linear motor 700 comprises a linearly displaceable actuator 710 which may be substantially similar in composition and manufacture to the rotor 320 shown in FIG. 3. The electric linear motor 700 also includes a stator 720 which may be substantially similar in composition and manufacture to the stator 310 shown in FIG. 3.

The actuator 710 also includes at least one magnet or magnetic component 730 (hereafter collectively referred to as the magnetic components 730) coupled to a structural portion 735. The stator 720 includes a substantially planar winding 740 and a magnetic portion 750 disposed proximate the magnetic components 730 such that energizing the winding 740 imparts linear motion to the actuator 710, possibly in the direction of the arrow 715. As in the embodiments discussed above, the magnetic portion 750 comprises a plurality of laminations.

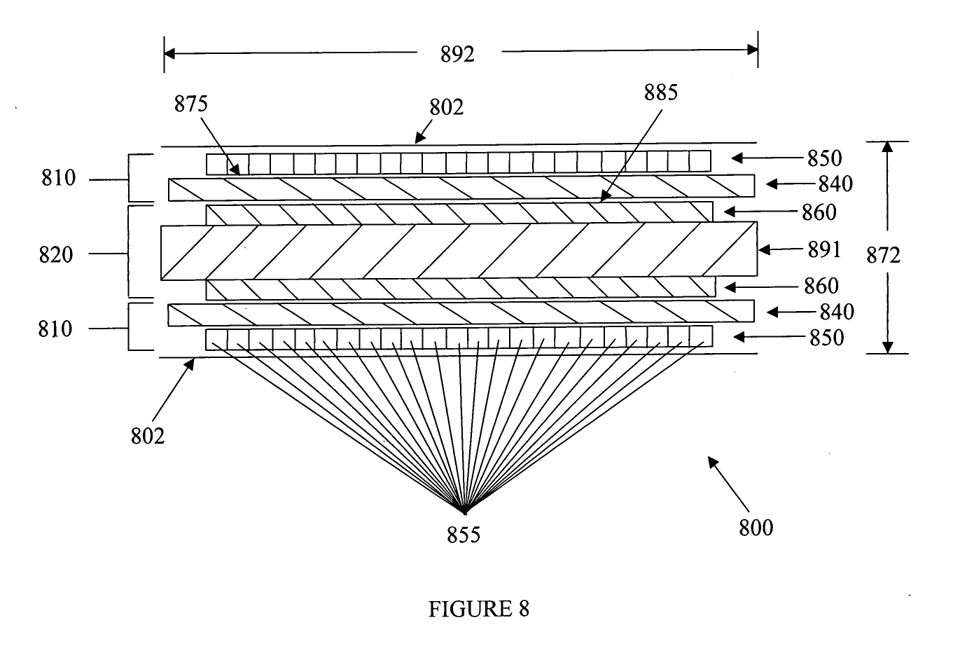

Referring to FIG. 8, illustrated is a side view of a section of a portion of a surgical instrument 800 constructed according to aspects of the present disclosure. The portion of the instrument 800 shown in FIG. 8 corresponds roughly to the motor assembly 12 portion shown in FIG. 2. The instrument 800 shown in FIG. 8 has a motor constructed similarly to that shown in FIG. 4. However, it will be recognized that any motor configuration described herein may be adapted for use with an instrument 800 as shown in FIG. 8. In FIG. 8, the motor comprises a stator 810 and a rotor 820. In the portion of the instrument 800 shown in FIG. 8, the rotor 820 is disposed within a cavity formed by the stator 810, such that the rotor 820 may rotate within the stator 310 in response to electric and/or magnetic fields generated by the stator 810 and/or the rotor 820. The rotor 820 comprises a structural portion 891 and a magnet portion 860. The stator 810 comprises a magnetically conductive portion 850 and a winding 840. The magnetically conductive portion 850 comprises a plurality of laminations 855. The laminations are of a thickness as discussed above. Preferably, there is an insulating layer 875 disposed between the magnetically conductive portion 850 of the stator 810 and the winding(s) 840. In addition, there may be a protective layer 885, such as a protective sleeve, between the winding(s) 840 and the magnetic portion(s) 860 of the rotor 820. A protective layer 885 may be desirable when the magnetic portion(s) 860 of the rotor 820 are brittle.

In FIG. 8, the stator 810 is fitted within a cavity formed by a surface 802 of the instrument 800. The outside diameter 872 formed by the surface 802 of the instrument 800 of the region of the instrument 800 housing the motor may be any size necessary to house an appropriate motor. However, as discussed above, the size of the instrument 800 is an important practical concern. Thus preferably, the outside diameter 872 of the instrument 800 in a region housing the motor is not substantially larger than that of currently available instruments. More preferably, the outside diameter 872 is substantially the same as or smaller than that of currently available surgical instruments. In an embodiment, the outside diameter 872 of the region housing the motor is less than about 30 mm. In an embodiment, the outside diameter 872 of the region housing the motor is less than about 25 mm. In an embodiment, the outside diameter 872 of the region housing the motor is less than about 20 mm. In an embodiment, the outside diameter 872 of the region of the instrument 800 housing the motor is less than about 16 mm. In an embodiment, the outside diameter 872 of this region is in the range of between about 15 mm and about 16 mm. In addition, it is preferred that the length 892 of the stator 810 is not substantially larger than that of motors used in currently available surgical instruments. More preferably, the length 892 of the stator 810 is substantially the same as or smaller than that of motors used in currently available surgical instruments. In an embodiment, the length 972 of the stator 810 is less than about 100 mm. In an embodiment, the length 972 of the stator 810 is less than about 60 mm. In an embodiment, the length 972 of the stator 810 is less than about 50 mm. In an embodiment, the outside diameter 872 of this region is in the range of between about 40 mm and about 50 mm.

The various aspects described above are applicable to, or may readily be adapted to, many electric motor applications, including embodiments not explicitly described or illustrated herein. For example, the electric motors shown in FIG. 3-6 may be 2-pole, 4-pole or otherwise configured motors. The aspects of the present disclosure are also applicable to motors having any operating speed or range thereof, although the benefits of such aspects will be better recognized at higher operating speeds. The aspects of the present disclosure are also applicable to motors of any size and capable of producing any amount of torque.

Although embodiments of the present disclosure have been described in detail, those skilled in the art should understand that they may make various changes, substitutions and alterations herein without departing from the spirit and scope of the present disclosure.

### EXAMPLE

The following example is provided to illustrate a specific embodiment of the invention only, and should not be construed as limiting the scope of the invention.

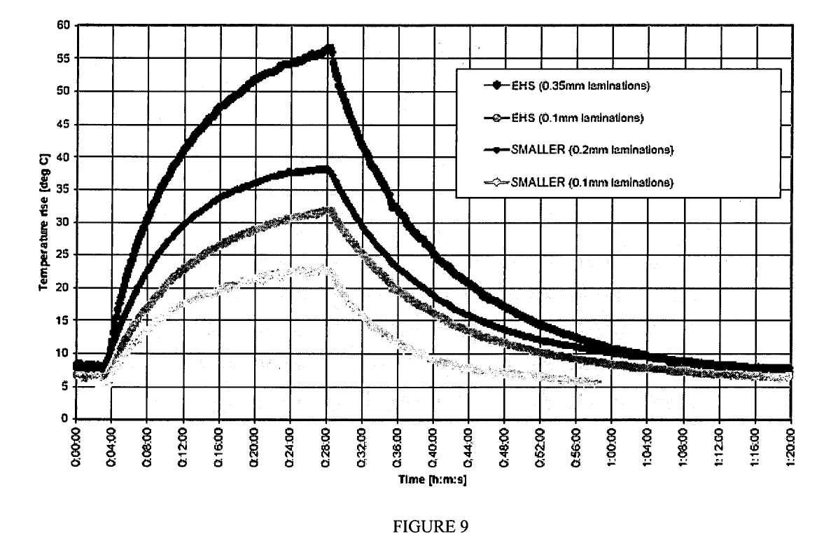

Surgical instruments based on Medtronic Midas Rex Model EHS high speed instrument, which has a diameter of about 21 mm in the portion housing the motor, and an instrument with smaller diameter, which has a diameter of 15.35 mm, were built. The instruments were built with motors having laminations of varying thickness. Instruments with laminations having a thickness of 0.1 mm were built and compared to Medtronic Midas Rex's currently available EHS high speed instrument, whose motor has stator laminations 0.35 mm thick. Motors having 0.1 mm thick stator laminations were housed in the housing of Midas Rex Model EHS high speed instrument. In addition, motors having 0.2 and 0.1 mm thick stator laminations were constructed and housed in a casing having an outside diameter of 15.35 mm (“SMALLER” as referred to in FIG. 9). The motors in the “SMALLER” instrument differed from the motors in EHS instruments. However, the motors in the “SMALLER” instruments differed essentially only with respect to their lamination thickness (i.e., 0.1 mm thick vs. 0.2 mm thick)

Motor output of Medtronic Midas Rex EHS-based instruments were measured for both the currently available 0.35 mm thick stator laminations and for 0.1 mm thick laminations. Both torque and power output at various speeds (rpm) were similar for instruments with motors having stator lamination thicknesses of 0.35 mm and 0.1 mm (data not shown). Thus, output performance was not adversely affected by reducing lamination thickness.

A thermal cross test was performed on EHS-based instruments having stator lamination thicknesses of 0.35 mm and 0.1 mm and on the “SMALLER” instruments having stator lamination thicknesses of 0.2 mm and 0.1 mm. The instruments were run at 70,000 revolutions per minute (rpm) for 25 min. Temperature measurements were taken just before the instruments were run (time 0:00:00), throughout the 25 min. period, and up to 100 min. after the start of the test (time 1:20:00). As shown in FIG. 9, the peak temperature rise of the EHS-based instrument with 0.1 mm thick stator laminations was about 25° C. less than that of the EHS-based instrument with 0.35 mm thick stator laminations (about 32° C. and about 57° C., respectively). In addition, the peak temperature rises of the SMALLER instruments with 0.2 mm thick stator laminations and 0.1 mm thick were about 38° C. and about 23° C., respectively.

As can be seen from the data presented in FIG. 9, an instrument having a smaller diameter in the region housing the motor has a more favorable temperature generation profile than an instrument having a larger outer diameter in a region housing the motor. For example, the SMALLER instrument having a diameter of 15.35 mm in the region housing the motor had a peak temperature rise of about 9° C. less than that of the EHS based instrument, which has a diameter of about 21 mm in the region housing the motor (about 23° C. and about 32° C., respectively). It is believed that the difference in heat generation between the two instruments having different diameters is due decreased iron losses in the smaller diameter instrument. Thus, maintaining a small diameter in a surgical instrument is not only desirable for ergonomic purposes, but also it is desirable from the aspect of heat generation.

FIG. 9 further shows that instruments having motors with thinner laminations exhibit more desirable heat generation profiles. As shown by the shape of the curves representing temperature over time in FIG. 9, the temperature increase of the instruments having thinner laminations (0.2 mm and 0.1 mm thick) begins to flatten out at about 25 minutes of operation. Thus, it is possible that much longer operation times would have little effect on increasing temperature further. As such, a threshold temperature beyond which the instrument becomes too hot for a surgeon to continue to use the instrument may not be reached with the instruments having thinner laminations. No breaks in surgery may be required with instruments with thinner stator laminations. In addition, curves for the SMALLER instruments with smaller diameters tend to flatten out more quickly than those with larger diameters (EHS). Generally, curves for the SAMLLER instruments flatten out after about 30 min of being run at 70,000 rpm, while the curves for the EHS instruments do not flatten out as quickly.

In light of the above, it is clear that surgeons will be provided significant advantages when using surgical instruments with electric motors having thinner laminations.

1. A surgical instrument, comprising:
a housing;
an electrical power source;
an output shaft extending from the housing;
a rotor coupled to the output shaft; and
a stator having:
a winding selectively connectable to the electrical power source; and
a magnetically conductive portion disposed about the rotor and comprising a plurality of laminations,
wherein one or more of the plurality of laminations has a thickness of less than about 0.25 mm;
wherein selectively connecting the electrical power source and the stator windings imparts rotary motion to the output shaft via the rotor.

2. The surgical instrument of claim 1, wherein each of the plurality of stator laminations has a thickness of less than about 0.25 mm.

3. The surgical instrument of claim 1, wherein the one or more laminations has a thickness of less than about 0.2 mm.

4. The surgical instrument of claim 2, wherein each of the laminations has a thickness of less than about 0.2 mm.

5. The surgical instrument of claim 1, wherein the one or more laminations has a thickness of less than about 0.15 mm.

6. The surgical instrument of claim 2, wherein each of the laminations has a thickness of less than about 0.15 mm.

7. The surgical instrument of claim 1, wherein the one or more laminations has a thickness of less than about 0.1 mm.

8. The surgical instrument of claim 2, wherein each of the laminations has a thickness of less than about 0.1 mm.

9. The surgical instrument of claim 1, wherein the one or more laminations has a thickness of about 0.2 mm.

10. The surgical instrument of claim 2, wherein each of the laminations has a thickness of about 0.2 mm.

11. The surgical instrument of claim 1, wherein the one or more laminations has a thickness of about 0.1 mm.

12. The surgical instrument of claim 2, wherein each of the laminations has a thickness of about 0.1 mm.

13. The surgical instrument of claim 1, wherein the housing, at least in a portion housing the stator, has an outer diameter of less than about 30 mm.

14. The surgical instrument of claim 13, wherein the housing, at least in a portion housing the stator, has an outer diameter of less than about 25 mm.

15. The surgical instrument of claim 14, wherein the housing, at least in a portion housing the stator, has an outer diameter of between about 20 mm and about 22 mm.

16. The surgical instrument of claim 14, wherein the housing, at least in a portion housing the stator, has an outer diameter of less than about 20 mm.

17. The surgical instrument of claim 13, wherein the housing, at least in a portion housing the stator, has an outer diameter of less than about 16 mm.

18. The surgical instrument of claim 13, wherein the housing, at least in a portion housing the stator, has an outer diameter of between about 15 mm and about 16 mm.

19. The surgical instrument of claim 13, wherein the stator has a length of less than about 100 mm.

20. The surgical instrument of claim 19, wherein the stator has a length of less than about 60 mm.

21. The surgical instrument of claim 20, wherein the stator has a length of less than about 50 mm.

22. The surgical instrument of claim 20, wherein the stator has a length in the range of between about 40 mm and about 50 mm

23. The surgical instrument of claim 10, wherein the housing, at least in a portion housing the stator, has an outer diameter of less than about 25 mm, and wherein the stator has a length of less than about 50 mm.

24. The surgical instrument of claim 12, wherein the housing, at least in a portion housing the stator, has an outer diameter of less than about 22 mm, and wherein the stator has a length of less than about 50 mm.

25. An electric motor for use in a surgical procedure, comprising:
a motor output member;
a driven member coupled to the motor output member; and
a driving member having a winding and a magnetically conductive portion disposed proximate the driven member such that energizing the driving member imparts motion to the driven member,
wherein the magnetically conductive portion comprises a plurality of laminations, and
wherein one or more of the laminations having a thickness of less than or equal to about 0.2 mm.

26. The electric motor of claim 25, wherein each of the laminations have a thickness of less than or equal to about 0.20 mm

27. The motor of claim 26, wherein each of the laminations have thickness of less than or equal to about 0.15 mm.

28. The motor of claim 25, wherein the motor is adapted for placement in an instrument having an outside diameter of less than about 25 mm.

29. The motor of claim 27, wherein the motor is adapted for placement in an instrument having an outside diameter of less than about 25 mm.

30. The motor of claim 29, wherein the stator has a length of less than about 50 mm.
